(19) United States
(12) Patent Application Publication
(10) Pub. No.: US 2011/0282352 A1
(43) Pub. Date: Nov. 17, 2011

(54) Trocar

(76) Inventors: Karlheinz Troendle, Geisingen (DE); Juergen Hartmann, Immendingen-Hattingen (DE)

(21) Appl. No.: 13/097,764

(22) Filed: Apr. 29, 2011

Related U.S. Application Data
(63) Continuation-in-part of application No. 13/096,549, filed on Apr. 28, 2011.

(30) Foreign Application Priority Data
Apr. 29, 2010 (DE) 102010016704.5-25

(51) Int. Cl. A61B 17/00

(52) U.S. Cl. 606/108

(57) ABSTRACT

A trocar with a movable flap for closing a trocar sleeve. The movable flap is automatically kept open with the aid of a locking mechanism.

### CROSS-REFERENCE TO RELATED APPLICATIONS

This application is a continuation-in-part of U.S. Ser. No. 13/096,549, filed Apr. 28, 2011, which claims priority of German Application No. 10 2010 016 704.5-25, filed Apr. 29, 2010.

### BACKGROUND OF THE INVENTION

The invention relates to a trocar having a movable flap for closing a trocar sleeve.

Trocars are used to introduce operating instruments for minimally invasive surgery, for example into the abdominal cavity of patients. For this purpose, a trocar sleeve is placed on the abdominal wall, a trocar pin (obturator) is pushed into the hollow trocar sleeve, an opening is made in the abdominal wall with the aid of the trocar pin, and the trocar sleeve is then introduced through the opening into the abdominal space. The trocar pin can then be withdrawn again from the trocar sleeve, and other surgical instruments can be inserted into the patient's abdominal space by way of the trocar sleeve engaged in the abdominal wall.

Most trocar sleeves can be closed in their longitudinal direction by a valve body, which can be opened by a medical instrument pushed into the trocar sleeve and which closes again automatically when the instrument is withdrawn. In practice, automatic flap valves have proven useful as valve bodies.

A flap valve of the type in question is described in DE 10 2005 042 892 A1. The valve body disclosed in the latter, and designed as a flap, is used to open and close an instrument or flow channel and is arranged so as to pivot on a pivot pin mounted in the housing. The flap is pretensioned in the direction of its closure position by a spring element, so as to avoid accidental opening of the valve body.

As is described in DE 43 062 05 C1, flaps of this kind for closing trocar sleeves can be made of metal, plastic or a combination of both.

The flaps described in DE 43 062 05 C1 can be opened only by medical instruments inserted into the trocar sleeve. A flap valve that can additionally be opened manually by a rotation element is described in DE 197 12 726 C1. The possibility of manually opening the valve flap has the advantage that sensitive medical instruments are not damaged by pressing open the flap.

The document WO 03/011153 A1 discloses a trocar head with a flap valve that can be opened by an external lever.

Valve mechanisms for closing trocar sleeves do not have to be constructed as a one-part flap but instead can also be composed of several flap elements, for example as described in EP 1 520 539 B1. The latter document describes a trocar with a closing mechanism composed of a plurality of semicircular flaps.

In all of these flap systems for closing trocar sleeves, the flaps are automatically closed again when the inserted medical instrument is withdrawn or when the actuating mechanism for opening the flap is let go. This automatic closing of the flap can be disadvantageous in some medical interventions. For example, when using complex instruments for minimally invasive surgery, when removing large volumes of tissue or when performing morcellation, flaps would be advantageous that do not automatically close again but instead can be automatically held open. The possibility of locking a trocar sleeve flap in the open state is mentioned in DE 296 07 146 U1. However, the latter does not give a technical example of such a locking mechanism.

The object of the invention is to overcome or at least minimize the disadvantages of the abovementioned prior art. The object of the invention is in particular to improve a locking mechanism for flaps in trocar sleeves.

### SUMMARY OF THE INVENTION

The object is achieved by a trocar according to the invention which has the advantage over the prior art, wherein a flap for closing a trocar sleeve not only can be pretensioned in the closing direction and thus automatically closed but can also be locked in a locking position and thus automatically kept open. In this way, certain medical interventions, for example morcellation or tissue removal, are made very much easier.

According to the invention, a trocar is provided with a trocar sleeve in which a movable flap is arranged for closing the trocar sleeve in the longitudinal direction. In preferred embodiments, the trocar sleeve is made up of more than one part and comprises a shaft tube, a housing, a main part, a tap attachment, a sealing cap receiver, and a sealing cap. Arranged on the flap is a locking mechanism which, when a minimum opening angle of the flap measured between the flap and the transverse axis of the trocar sleeve is reached, brings the flap automatically to a locking position and thus keeps it open. In the preferred embodiment, the flap, in the locking position, is displaced slightly along the transverse axis of the trocar sleeve in relation to a closed position. In typical embodiments, the locking in the locking position does not take place automatically but by manual displacement. Advantageous embodiments are characterized in that the flap is not pushed automatically into a locking mechanism but is held automatically in an open position.

In the preferred illustrative embodiment, the minimum opening angle is more than 60 degrees and less than 20 degrees, preference being given to minimum opening angles of more than 70° and less than 110° , particular preference being given to minimum opening angles of more than 80° and less than 100° , of which minimum opening angles of more than 85° and less than 95° are particularly preferred.

In the preferred illustrative embodiment, an energy reservoir, preferably a spring, is arranged between the trocar sleeve and the flap, for example a compression spring or a tension spring, which presses the flap automatically into the locking position when the minimum opening angle is reached. Energy reservoir or spring is understood here as a structural part that yields under a load and returns to the original configuration when the load is removed, i.e. acts elastically.

In advantageous embodiments, a closing spring is arranged between the trocar sleeve and the flap and pretensions the flap in the closing direction. This closing spring brings the flap automatically to its closure position if the flap is not being held in the locking position or the open position.

In the preferred embodiment, an actuating mechanism for manually pivoting the flap is arranged on the trocar sleeve. A hinge pin is preferably arranged between flap and actuating mechanism. In preferred embodiments, the actuating mechanism comprises a lever. By pressing on the actuating mechanism, the flap can preferably be pivoted steplessly in the direction of the open position. However, this pivoting of the flap is preferably possible not only with the aid of the actuating mechanism but also by means of an instrument inserted into the trocar sleeve.

In preferred embodiments, the actuating mechanism, preferably the lever, comprises a notch in the shape of a finger pad. This has the advantage of avoiding the finger slipping off during actuation of the actuating mechanism.

In the preferred embodiment, the locking mechanism comprises a locking slit into which the flap is at least partially pushed when the minimum opening angle is reached. This locking slit preferably extends substantially parallel to the longitudinal axis of the trocar sleeve. Such a locking slit has the advantage that the flap can be held very simply in the locking position. In advantageous embodiments, the locking slit does not extend substantially parallel to the longitudinal axis of the trocar sleeve but instead has another orientation in relation to the longitudinal direction of the trocar sleeve.

In typical embodiments, when the minimum opening angle is reached, the flap is not pushed automatically into a locking slit but is instead held automatically in an open position. In advantageous embodiments, it is held in the open position by a blocking bush, which slides at least partially over or into the flap when the minimum opening angle is reached.

In particularly advantageous embodiments, the flap is simultaneously brought into a locking slit and held securely by a blocking bush.

In advantageous embodiments, a signaling means for indicating the locking is arranged on the trocar sleeve. In particularly advantageous embodiments, the signaling means comprises a colored ring. This has the advantage that the operator can easily see whether the flap is locked or freed.

During typical use of the trocar, the flap is opened steplessly either by manual actuation of the actuating mechanism or by insertion of a medical instrument. As soon as the flap reaches the minimum opening angle, it is automatically moved at least partially into the locking slit. There, the flap is then held in the locking position. The flap is then preferably released from the locking position, by actuation of the actuating mechanism, when an opened flap is no longer necessary. The flap is preferably released by the actuating mechanism being pressed substantially in the transverse direction of the trocar sleeve. In advantageous embodiments, another type of actuation of the actuating mechanism, e.g. pulling, turning or lifting, leads to the flap being released from the locking position.

In an advantageous method for locking the flap, the flap is first of all successively opened from the closure position by manual actuation of the actuating mechanism or by an instrument inserted into the trocar sleeve. As soon as the minimum opening angle is reached, a blocking bush slides at least partially over or into the flap. In this way, the flap can now no longer close and is held in an open position. The release of the flap from the open position is typically effected by actuation of the actuating mechanism, preferably by pressing the actuating mechanism substantially in the transverse direction of the trocar sleeve. In advantageous embodiments, another type of actuation of the actuating mechanism, e.g. pulling, turning or lifting, leads to the flap being released from the locking position.

### BRIEF DESCRIPTION OF THE DRAWINGS

The invention is explained in more detail below with reference to schematic drawings in which:

FIG. 1 shows an outside view of a trocar sleeve with a lever,

FIG. 2 shows a cross section through the trocar sleeve according to FIG. 1 along the line II-II at the height of the lever and of a locking mechanism,

FIG. 3 shows a cross section through the trocar sleeve at the height of the lever and of the locking mechanism when the flap is open, and

FIG. 4 shows a cross section through the trocar sleeve at the height of the lever and of the locking mechanism when the flap is open, in an embodiment with a blocking bush,

FIG. 5 shows a schematic side view of a partial area of the trocar sleeve according to the invention, with the flap locked in the open position,

FIG. 6 shows a schematic side view as in FIG. 5, with the flap closed.

### DETAILED DESCRIPTION

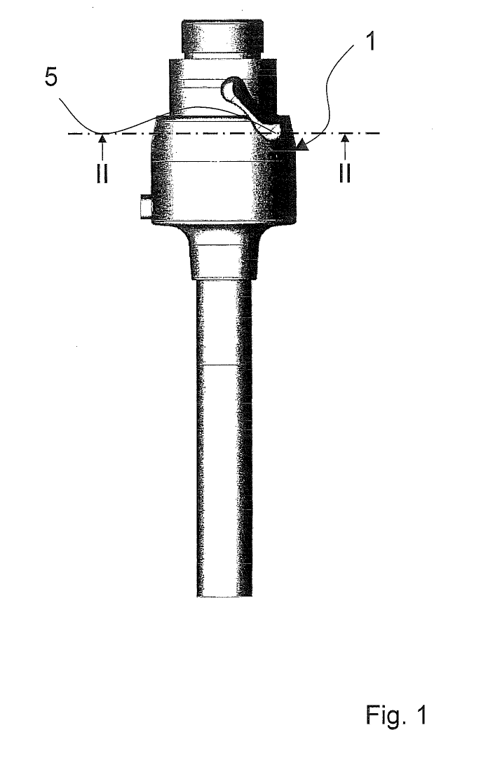

FIG. 1 shows a trocar sleeve 1 of a trocar (not described in any more detail), with an actuating mechanism 5 that is designed as a lever and has a notch shaped like a finger pad.

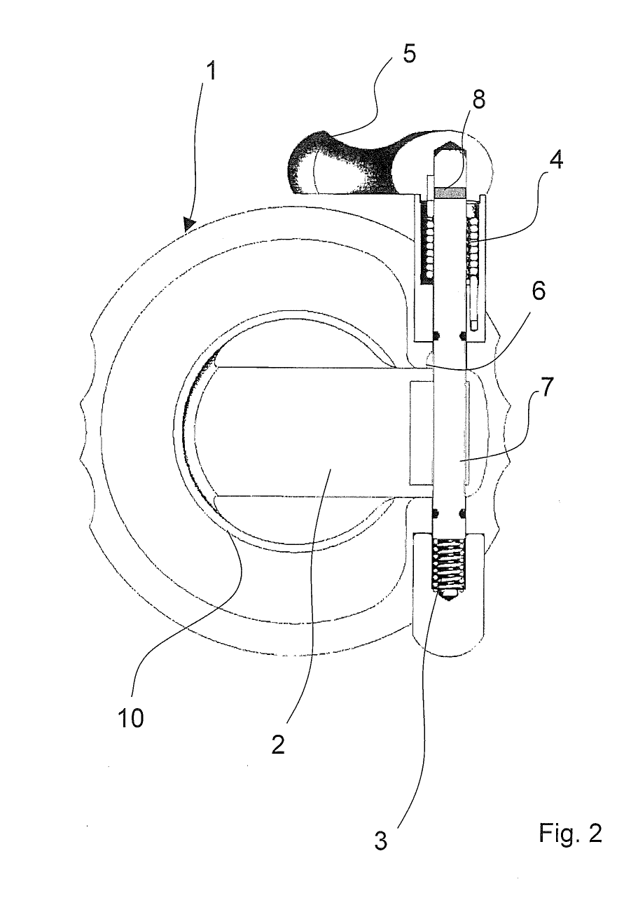

FIG. 2 shows a cross section through the trocar sleeve 1, in which a flap 2 for closing the trocar sleeve 1 can be clearly seen. The flap 2 is shown in its closure position. In this closure position, it closes a passage through the trocar sleeve by bearing on a sealing ring 10. The flap 2 is connected to the actuating mechanism 5 via a hinge pin 7. A closing spring 4 pretensions the hinge pin 7 and flap 2 against the trocar sleeve in the closing direction of the flap 2. By actuation of the actuating mechanism 5, the flap 2 can be opened gradually and steplessly.

When the flap reaches a minimum opening angle, it is pressed by a compression spring 3 automatically into a locking slit 6. The flap 2 is then automatically locked there until the locking mechanism is released via the actuating mechanism 5.

A signaling means 8 is arranged on the hinge pin 7 and is designed as a colored ring that is visible on the outside of the trocar sleeve 1 when the flap 2 is opened. This shows the user whether or not the flap 2 is locked.

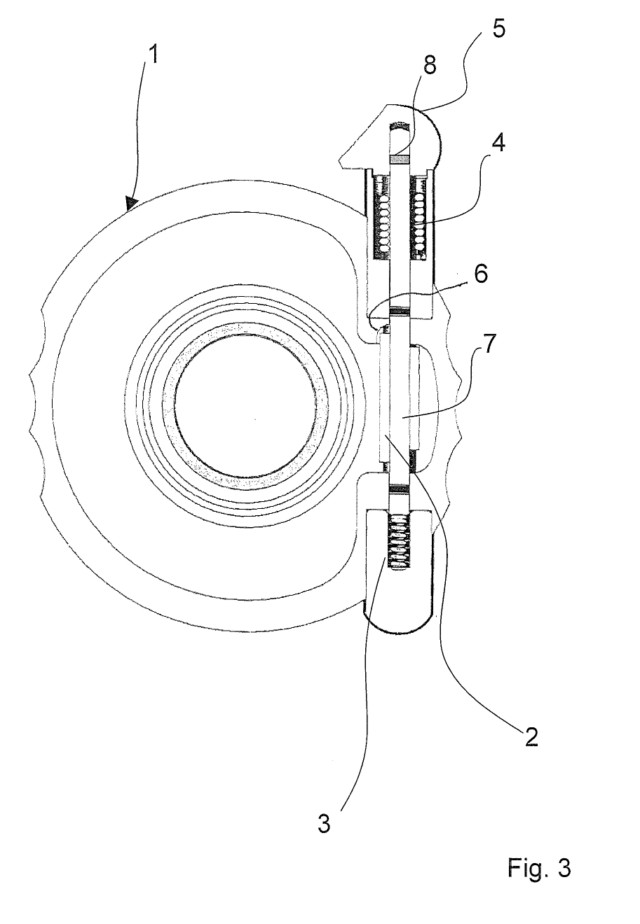

FIG. 3 shows the same view as FIG. 2, but the flap 2 in FIG. 3 is open. It can be seen from FIG. 3 that the flap 2 is driven partially into the locking slit 6 and is automatically held there. Only when the actuating mechanism 5 is pressed in the transverse direction of the trocar sleeve 1 can the flap 2 be pressed back out of the locking slit. The force exerted by the compression spring 3 must be counteracted to do so.

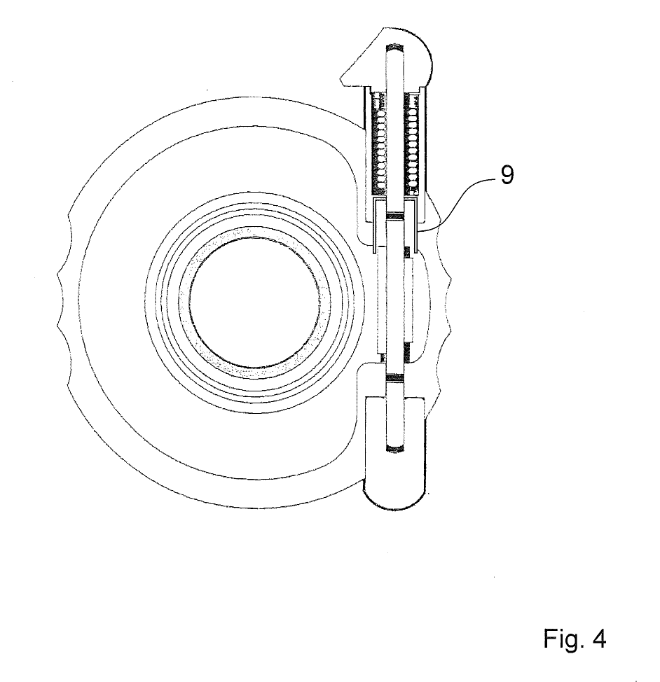

FIG. 4 shows another illustrative embodiment in which the flap 2 is held in an open position by a blocking bush 9, which is pushed partially over the flap 2.

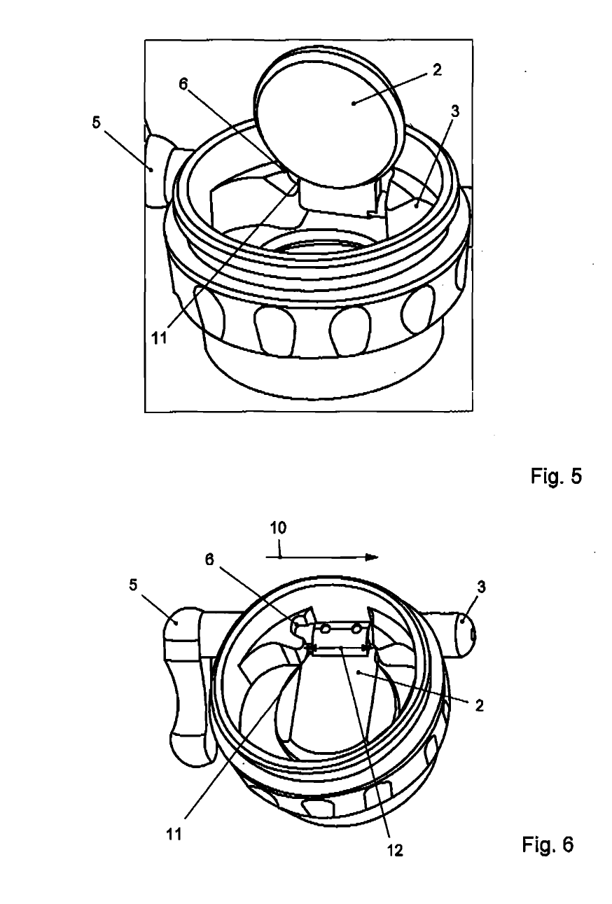

The flap 2 is shown in an open position in FIG. 5. It can easily be seen how, in this open position, the flap 2 is pushed into the locking slit 6. This pushing-in is achieved, on the one hand, by actuation of the lever 5 and, on the other hand, by the compression spring 3. Although the compression spring 3 is not depicted in FIG. 3, it is indicated by a reference sign.

FIG. 6 shows a closed flap 2. From the open position, which was shown in FIG. 5, the user gets to the closed position, as shown in FIG. 6, by pressing the lever 5 in the direction of an arrow 10 counter to the force of the compression spring 3 and additionally turning said lever. In this way, a base area 11 of the cover 2 is released from the locking slit 6. The base area 11 is designed in such a way that it is pivotable in a recess 12 of the trocar sleeve 1. When the flap 2 is opened again, the base area 11 is pivoted through about 70° to 90° by rotation of the lever. After the base area 11 has passed through the recess 12, the compression spring 3 presses the base area 11 or rather the cover 2 into the locking slit 6.

The invention is not limited to the illustrative embodiments described above. The scope of protection is defined instead by the claims.

1-10. (canceled)

11. Trocar comprising:
a trocar sleeve (1), which comprises at least one structural element;
a movable flap (2) arranged in the trocar sleeve (1) for closing the trocar sleeve in the longitudinal direction of the trocar sleeve (1);
a locking mechanism assigned to the movable flap (2), wherein,
when a minimum opening angle measured between the flap (2) and a transverse axis of the trocar sleeve (1) is reached, the flap (2) is held automatically in an open position by the locking mechanism or brought to a locking position.

12. Trocar according to claim 11, wherein the minimum opening angle is greater than 60 degrees and less than 120 degrees.

13. Trocar according to claim 11, wherein a compression spring (3), which loads the flap (2) against the trocar sleeve (1), is arranged such that the flap (2) is pressed automatically into the locking position when the minimum opening angle is reached.

14. Trocar according to claim 11, wherein located between the trocar sleeve (1) and the flap (2) is a closing spring (4) arranged such that the flap is pretensioned in a closing direction.

15. Trocar according to claim 11, wherein an actuating mechanism (5) for pivoting the flap (2) is arranged on the trocar sleeve (1), and a hinge pin (7) is arranged between the flap (2) and the actuating mechanism (5).

16. Trocar according to claim 11, wherein the locking mechanism comprises a locking slit (6).

17. Trocar according to claim 11, wherein the locking mechanism comprises a blocking bush (9) arranged to slide at least partially over the flap (2) when the minimum opening angle is reached.

18. Trocar according to claim 11, wherein a signaling means (8) for indicating locking is arranged on the trocar sleeve (1).

19. Method for locking and releasing a flap (2) in a trocar sleeve (1) including a locking mechanism having a locking slit, comprising the steps of:
manually actuating an actuating mechanism (5) in order to open the flap (2);
bringing the flap (2) at least partially into the locking slit (6) of a locking mechanism as soon as a minimum opening angle is reached wherein the minimum opening angle is greater than 60 degrees and less than 120 degrees;
holding the flap (2) in a locking position; and
releasing the flap (2) from the locking position by actuation of the actuating mechanism (5).

20. Method for locking and releasing a flap (2) in a trocar sleeve (1) including a locking mechanism having a blocking bush arranged to slide at least partially over the flap when a minimum opening angle is reached, comprising the steps of:
manually actuating an actuating mechanism (5) in order to open the flap (2);
sliding the blocking bush (9) at least partially over the flap (2) as soon as a minimum opening angle is reached wherein the minimum opening angle is greater than 60 degrees and less than 120 degrees;
holding the flap (2) in the open position; and
releasing the flap (2) from the open position by actuation of the actuating mechanism (5).
